(19) United States
(12) Patent Application Publication
(10) Pub. No.: US 2024/0117403 A1
(43) Pub. Date: Apr. 11, 2024

(54) METHODS FOR DETECTING MICROORGANISMS

(71) Applicant: Spectacular Labs, Inc., Richmond, CA (US)

(72) Inventors: Okhtay Montazeri, Richmond, CA (US); Yuan-Sheng Fang, Oakland, CA (US)

(73) Assignee: Spectacular Labs, Inc., Richmond, CA (US)

(21) Appl. No.: 18/392,764

(22) Filed: Dec. 21, 2023

Related U.S. Application Data
(63) Continuation-in-part of application No. 17/684,403, filed on Mar. 1, 2022.

(51) Int. Cl. C12Q 1/04; C12Q 1/686

(52) U.S. Cl. CPC C12Q 1/04; C12Q 1/686

(57) ABSTRACT

A method, comprising placing a biological sample into a container for detecting a presence or an absence of at least one microorganism via testing of the biological sample within the container as directed by a processor system substantially in accordance with software executing on the processor system. The method further comprises performing at least one analysis on the biological sample within the container in an automated manner directed by the processor system. The method further comprises detecting the presence or the absence of the at least one microorganism as a result of the at least one analysis performed on the biological sample directed by the processor system.

### CLAIM FOR PRIORITY

This application is a Continuation-In-Part of U.S. patent application Ser. No. 17/684,403, filed Mar. 1, 2022, titled “METHODS FOR DETECTING MICROORGANISMS,” and which is incorporated by reference in its entirety.

### TECHNICAL FIELD

This description relates generally to methods for detecting microorganisms such as bacteria, viruses, or fungi.

### BACKGROUND

Microorganisms are abundant in the environment, and a large portion of the cells that exist in larger animals, such as humans, are “external” microorganisms which do not share the same genetic material as the “host” species. Many of the microorganisms which exist in the environment or even within another “host” are either benign or beneficial. Identifying the good bacteria, such as probiotics, and healthy gut flora may be useful for the overall health of the “host,” and that of the environment, as the growth and abundance of “good” microorganisms can hinder or suppress the growth of “bad” or pathogenic microorganisms. However, in certain circumstances, there may be special interest to detect the presence of pathogenic strains in food or surfaces for certain applications.

Pathogens are infectious microorganisms such as bacteria, viruses, or fungi. In food and in the environment, the presence of pathogens can create significant health hazards. For example, food contaminated with Listeria, Salmonella or Escherichia coli pose a serious threat to human health. Other pathogens, such as tuberculosis, pose a serious threat to livestock animals. Every year, almost one sixth of the people in North America become ill as a result of exposure to foodborne pathogens.

Current methods for detecting the presence of microorganisms in food and in the environment have limitations which allow microorganisms to go undetected. Current methods are not automated and at the same time easy-to-use, thus requiring trained operators to perform one or more steps. Also, current methods may not accurately detect the presence of viable organisms, especially if a sample contains a low number of microorganisms. Furthermore, current methods are expensive, often requiring specialized laboratory environments for their operations which requires samples to be transported to third-party laboratories, and as a result, there may be a large lag time between sampling and results.

The background description provided here is for the purpose of generally presenting the context of the disclosure. Unless otherwise indicated here, the material described in this section is not prior art to the claims in this application and are not admitted being prior art by inclusion in this section.

### SUMMARY

One method of microorganism detection includes selection of test parameters and preparation of a sample based on at least one of the test parameters. The method includes conducting tests on the sample based on the test parameters and providing test results and related data, wherein the test results indicate the presence or absence of the microorganism. The method includes analyzing test results and related data and providing qualitative and quantitative assessments of test results and related data. The method includes adjusting the test parameters to optimize the test parameters. The method includes repeating the tests with the adjusted test parameters and adjusting the test parameters based on analyses of the test results and the related data to optimize the test parameters.

Optimizing the test parameters includes increased reliability of detecting the presence or absence of the microorganism, reduction of an average time to obtain test results, and/or reduction of cost of reagents used to perform the tests.

This method includes enriching the sample by diluting the sample with a liquid enrichment medium and incubating the diluted sample to allow levels of the microorganism to increase. The method includes lysing the enriched sample with a lysis solution to break down cells of the microorganism and to release nucleic compounds of the microorganism. The method includes amplifying the nucleic compounds to increase the number or the length of a chain of the nucleic compounds. The method includes detecting the presence or the absence of the microorganism by assaying the sample.

A machine-readable storage media includes machine-readable instructions stored thereon, that when executed, cause one or more machines to perform a method for detecting a microorganism in a sample. The method includes selecting test parameters and preparing a sample based on at least one of the test parameters and conducting tests on the sample based on the test parameters. The method includes providing test results and related data, wherein the test results indicate the presence or an absence of the microorganism. The method includes analyzing the test results and the related data and providing qualitative and quantitative assessments of the test results and the related data. The method includes adjusting the test parameters to optimize the test parameters. The method includes repeating the tests with the adjusted test parameters and adjusting the test parameters based on analyses of the test results and the related data to optimize the test parameters.

A system for detection of a microorganism includes a first module having interfaces configured to receive a sample and test parameters. The first module is operable to conduct tests on the sample and provide test results and related data. The test results indicate the presence or an absence of the microorganism in the sample. The system includes a second module having an interface configured to receive the test results and related data. The second module is operable to analyze the test results and related data and provide qualitative and quantitative assessments of the test results and related data. The system includes a third module having an interface configured to receive the qualitative and quantitative assessments and in response adjust the test parameters to optimize the test parameters. The adjusted test parameters are provided to the first module for subsequent tests.

A method of detecting a microorganism includes selecting test parameters and enriching a sample with at least one of the test parameters. The method includes conducting tests on the enriched sample based on the test parameters and providing test results and related data, wherein the test results indicate the presence or an absence of the microorganism. The method includes analyzing the test results and the related data and providing qualitative and quantitative assessments of the test results and the related data. The method includes adjusting the test parameters to optimize the test parameters. The method includes repeating the tests with the adjusted test parameters and adjusting the test parameters based on analyses of the test results and the related data to optimize the test parameters.

### BRIEF DESCRIPTION OF THE DRAWINGS

The embodiments of the disclosure will be understood more fully from the detailed description given below and from the accompanying drawings of various embodiments of the disclosure, which, however, should not be taken to limit the disclosure to the specific embodiments, but are for explanation and understanding only.

FIG. 1 and FIG. 2 illustrate block diagrams of systems for detection of microorganisms in accordance with some examples.

FIG. 3 illustrates a flow diagram of a method of detection in accordance with at least one example.

FIG. 4 illustrates a flow diagram of a method of conducting tests in accordance with at least one example.

FIG. 5A illustrates a first embodiment of a portable bioanalysis station, in accordance with at least one example.

FIG. 5B illustrates a second embodiment of a portable bioanalysis station, in accordance with at least one example.

FIG. 5C illustrates a third embodiment of a portable bioanalysis station, in accordance with at least one example.

FIG. 6 illustrates a block diagram of an exemplary control configuration for a portable bioanalysis station, in accordance with at least one example.

FIG. 7 illustrates a processor system in accordance with at least one example.

The same reference numerals or other reference designators are used in the drawings to designate the same or similar (by function and/or structure) features.

### DETAILED DESCRIPTION

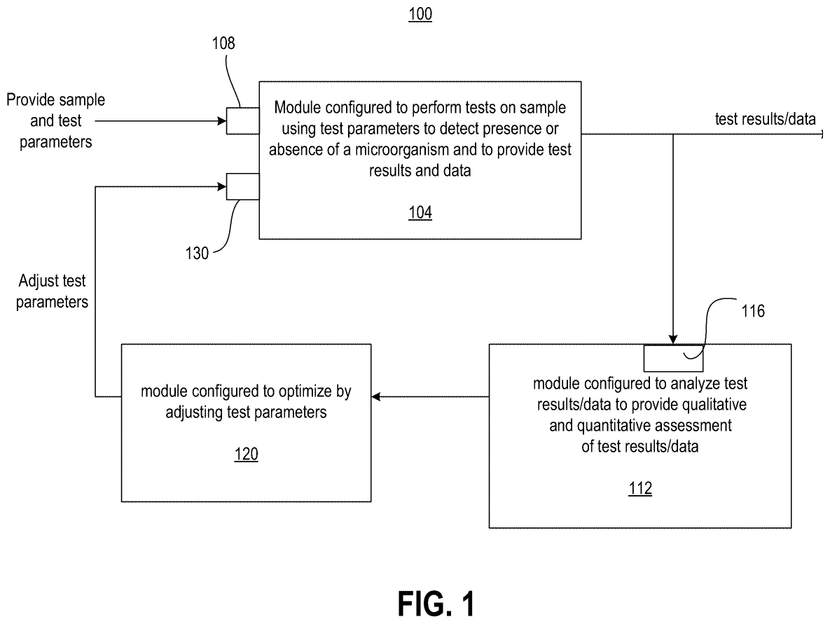

FIG. 1 illustrates a block diagram of a system 100 for detection of microorganisms in accordance with at least one example. Microorganisms may be any kind of bacterial, viral, microbial, fungal, or parasitic pathogens that are capable of multiplication.

In the following description, system 100 (and system 200 described subsequently) provides software and apparatus hardware that enables a self-contained and automated miniaturized laboratory for rapid determination of pathogenic and benign microorganisms. Software included in system 100 comprises subroutines and algorithms, as described in the various method embodiments introduced below, that direct automated laboratory manipulations of biological sample preparation and sample transfer without human intervention, as well as reagent transfer by apparatus hardware. Software includes subroutines that direct reaction promotion by heating of reagents to enable reactions necessary to generate target analytes, and then detection of such target analytes by apparatus hardware. As the miniaturized laboratory is self-contained, operator intervention or supervision is circumvented by fully robotic operation of the apparatus for analytical runs that may have been optimized prior to standardization. Such robotic operation relies solely on software control which are based on methods described herein, therefore, in at least one example, no human intervention is involved in analysis methods described herein with the exception of initial introduction of a biological sample to the apparatus.

In addition, optimization procedures are described herein, whereby parameters of temperature, time, nutrient types, and concentrations, etc., are tested and best combination are discovered by statistical procedures such as Bayesian optimization, as described below. System 100 provides software algorithms that implement statistical analyses such as Bayesian optimization, which use digital computation for efficient characterization of enrichment and analysis to arrive at accurate identification of microorganisms in a short time period, for example, within 48 hours or less. The optimized parameters may be accepted and stored in software subroutines for execution during analytical runs.

In at least one example, system 100 includes a module or platform 104 which includes one or more inputs or interfaces 108 configured to receive a sample and test parameters. The sample may be solid, semi-solid, or liquid which may be contaminated with a microorganism. Any sampling method is encompassed, if it is suitable to acquire a sample of the microorganism of interest. A sample may be acquired by excision, blotting, swabbing, sponging etc. Samples may include, but are not limited to: meat, poultry, fish, produce, juices, dairy products, dry goods, raw and processed foods, tissue, urine, fecal matter, water, wastewater, soil, or surface samples. In at least one example, system 100 may include a plurality of modules or platforms 104 that are located at different physical locations.

The test parameters include, but are not limited to: reagents, supplements, diluents, and other chemicals which are added to the sample; concentration and ratios of the reagents, supplements, diluents, and other chemicals; temperature range; and durations (e.g., incubation period, enrichment period).

In at least one example, interface 108 may be input ports configured to receive one or more cartridges or containers which may hold the sample, the reagents, the supplements, the diluents, and other chemicals which are used for the tests. In at least one example, module or platform 104 performs a series of tests to detect the presence or absence of the microorganism in the sample. The tests may be performed in an order, however, the order of the tests may be changed depending on the sample or other criteria. Based on the tests, in at least one example, module or platform 104 provides test results and related data which indicate the presence or absence of the microorganism of interest in the sample. For example, the test results may indicate the presence of the microorganism by a presumptive positive result and may indicate the absence of the microorganism by a presumptive negative result. The test results may also include the test parameters such as, but not limited to: reagents, supplements, diluents, and other chemicals which were added to the sample; such as concentration and ratios of the reagents, supplements, diluents, and other chemicals; raw data collected during the test; temperature range; durations (e.g., incubation period, enrichment period, total time for completion of the detection).

In at least one example, system 100 includes a module or platform 112 having inputs or interfaces 116 configured to receive the test results and related data. In at least one example, module or platform 112 analyzes the test results and related data and provides qualitative and/or quantitative assessments of the test results and related data. As more tests are performed by module or platform 104, at least one example, module 112 repeats analysis of the test results and related data and provides qualitative and/or quantitative assessments.

In at least one example, a qualitative assessment of the test results and related data includes detecting the presence or absence of the microorganism in the sample as well as the reliability or accuracy of the detection. The quantitative assessment of the test results and related data may include an assessment of the test parameters used in the test.

In at least one example, system 100 includes a module or platform 120 which has an input or interface configured to receive the qualitative and/or quantitative assessments of the test results and related data. In response to the qualitative and/or quantitative assessments, module, or platform 120 adjusts or updates one or more test parameters to optimize or modify the test parameters and improve performance of system 100.

In at least one example, module or platform 104 includes an input or interface 130 configured to receive the adjusted or updated test parameters. In at least one example, module or platform 104 performs subsequent tests using the adjusted or updated test parameters and provides test results and related data which are again analyzed by module or platform 112 to provide qualitative and/or quantitative assessments. In response, at least one example, module or platform 120 adjusts the test parameters to further optimize or modify the test parameters and improve performance of the system 100. In at least one example, the foregoing process is repeated for each test, and based on the qualitative and/or quantitative assessments, the test parameters are adjusted to optimize or modify the test parameters and improve the performance of system 100.

In at least one example, module or platform 120 may vary the concentration and ratios of the reagents, supplements, diluents, and other chemicals and may vary the temperature and durations (e.g., incubation period, or enrichment period) for optimization of the test parameters to improve performance of the system 100. In at least one example, module or platform 120 may adjust the test parameters to increase reliability of the detection of presence or absence of the microorganism. Other parameters may be varied for optimization. In at least one example, module 120 may adjust the test parameters to reduce the average time required to obtain test results and/or reduce the amount of reagents, supplements, and other chemicals to reduce the cost.

In at least one example, modules 112 and 120 include an algorithm for optimizing various test parameters, criteria, or benchmarks. In at least one example, the criteria or benchmarks can include, but are not limited to: the average time to obtain test results, the average cost of reagents, supplements and other chemicals used to perform each test, or sensitivity of the detection of the microorganism of interest. In at least one example, the algorithm may recommend if simultaneous tests for different nucleic sequences should be run as opposed to individual tests. In at least one example, the algorithm may also recommend if tests should be performed at the onset of a specific event. In at least one example, the algorithm may also recommend if the test sample should be preserved or transmitted to an outside laboratory for further testing. In at least one example, the algorithm may issue a certification of the analysis if required conditions are met.

In at least one example, module or platform 104 includes an input 130 for receiving the adjusted or updated test parameters. In at least one example, module or platform 104 performs subsequent tests using the adjusted or updated test parameters and provides test results and related data.

In at least one example, module 104 is configured to detect if the sample contains a specific nucleic sequence. Milk from a dairy producer may be tested for the presence of Campylobacter or a sample of ground meat may be tested for the presence of E. coli bacteria.

In at least one example, modules 112 and 120 can be combined into a single module which can be configured to perform the functionalities of both modules 112 and 120.

In at least one example, modules or platforms 112 and 120 may be placed at various locations. In at least one example, modules or platforms 112 and 120 may be a secure host service that can be hosted on an external server or on a cloud server. In at least one example, module 104 may transmit test results and related data to an external server or a cloud server. In at least one example, the external server or a cloud server may analyze the test results and related data and provide quantitative and/or qualitative assessments of the test results and related data and adjust or vary the test parameters to optimize or modify the test parameters and improve performance of system 100. In at least one example, there may be multiple external servers or cloud servers that analyze the test results and related data. For example, different external servers or cloud servers may each analyze only parts of the test results and related data and based on their respective analysis provide qualitative and/or quantitative assessments which may be shared among the multiple external servers or cloud servers. The qualitative and/or quantitative assessments may be transmitted back to module 104. In at least one example, the system includes multiple modules 104 (i.e., multiple test modules 104) that are located at different physical locations. The external server may transmit the qualitative and/or quantitative assessments and adjusted test parameters back to the multiple test modules 104. In response, the multiple test modules may conduct subsequent tests using the adjusted test parameters. In at least one example, only a subset of the multiple test modules 104 may transmit the test results and related data to the external servers or the cloud servers which analyze the test results and related data and provide qualitative and quantitative assessments.

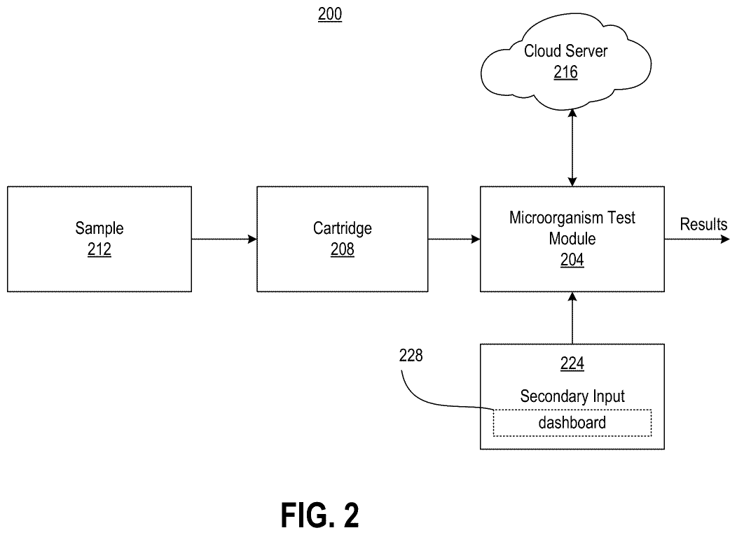

FIG. 2 is a block diagram of a system 200, in accordance with at least one example. In at least one example, system 200 implements system 100 illustrated in FIG. 1. In at least one example, system 200 includes a module or platform 204 (microorganism test module 204) configured to run tests to detect the presence or absence of microorganism in a test sample. In at least one example, module or platform 204 may be in the form of a box that has one or more receptacles or bins for receiving one or more cartridges or containers 208. In at least one example, cartridges 208 may be pre-loaded with reagents, supplements, diluents, and other materials. In at least one example, a sample 212, which may be contaminated with the microorganism, can be loaded into cartridge 208, and cartridge 208 may then be loaded into module or platform 204.

In at least one example, system 200 includes a module 216 (e.g., cloud server 216, external server, local computing machine, mobile device, etc.) which may be placed at various locations. In at least one example, module 216 may be a secure host service that can be hosted on an external server or on a cloud server. In at least one example, platform 204 may transmit the test results and related data to module 216. In at least one example, module 216 is an external server or a cloud server. In at least one example, the external server or cloud server may include algorithms for analyzing the test results and related data. In at least one example, the algorithm analyzes the test results and related data, and based on the analysis provides quantitative and/or qualitative assessments of the test results and related data. Based on the qualitative and/or quantitative assessments, in at least one example, the algorithm adjusts the test parameters to optimize or modify the test parameters and improve performance of system 200. In at least one example, the qualitative and/or quantitative assessments and the adjusted or updated test parameters are transmitted back to module or platform 204. In at least one example, the adjusted or updated test parameters are used by module or platform 204 to run subsequent tests. As such, the test results and related data are used by system 200 to optimize or modify subsequent test parameters and improve performance of system 200.

In at least one example, system 200 includes a user interface 224 to enable a user to configure module or platform 204. A user may provide secondary inputs to module platform 204. For example, a user may set various test parameters (e.g., temperature, duration, ratios) via user interface 224. In at least one example, user interface 224 may include a dashboard 228 configured to interact with a user. In at least one example, dashboard 228 may be configured to receive results of the analysis and visualize test results. In at least one example, dashboard 228 may be configured to track test status, results, and test schedules.

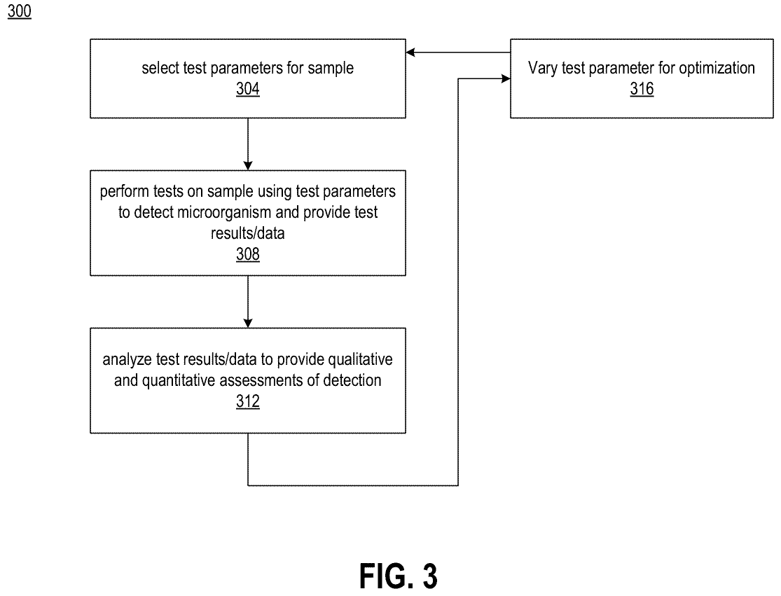

FIG. 3 illustrates a flow diagram of a method of detection 300, in accordance with at least one example. In block 304, test parameters are selected for a sample. The order of various blocks shown here can be modified. For example, some blocks may be performed simultaneously. In some embodiments, the various blocks shown here can be implemented in hardware, software, or a combination of them. The sample (e.g., sample 212) may be solid, semi-solid, or liquid which may be contaminated with a microorganism. The test parameters include, but are not limited to: reagents, supplements, diluents, and other chemicals which are added to the sample; concentration and ratios of the reagents, supplements, diluents and other chemicals; temperature range; and durations (e.g., incubation period, enrichment period). These parameters can be pre-loaded into cartridge 208, in accordance with at least one example.

In block 308, tests are performed on the sample using the test parameters and test results and related data are provided. For example, test module 204 executes a process discussed herein to detect the microorganism of interest. The test results indicate the presence or absence of the microorganism in the sample. For example, the test results may indicate the presence of the microorganism by a presumptive positive result and may indicate the absence of the microorganism by a presumptive negative result. The test results and related data may include the test parameters such as, but not limited to: reagents, supplements, diluents, and other chemicals which were added to the sample; concentration and ratios of the reagents, supplements, diluents, and other chemicals; raw data collected during the test; temperature range; and durations (e.g., incubation period, enrichment period, total time for completion of the detection).

In at least one example, enrichment time may be optimized by varying enrichment parameters, such as temperature, pH, supplement concentrations, etc. An optimization of enrichment time may include varying experimental parameters that lead to reduction of enrichment time. To explore the entire parameter space comprising multiple parameters, an experimental design approach may be employed, whereby experimental data may be interpreted by Bayesian inference. In at least one example, a single parameter or a combination of parameters may be found that can lead to a reduction of enrichment time by acquiring results from successive experiments and updating a probability finding the key parameter(s).

In at least one example, a model may be built from an initial set of N observations of how enrichment time varies with changes to selected enrichment parameters. In at least one example, enrichment parameters may be chosen from a library of sampling protocols, stored within servers or modules such as module 104 described above. The model may then compute an acquisition function that may inform the user of the best subsequent experiment to perform. After this experiment is performed, the acquired observations may be added to the initial set of N observations as the N+1th observation. The N+1th observation is input to the algorithm pipeline to obtain a subsequent set of experimental conditions. This process may be iterated until a reduction of enrichment time may meet a defined criterion, for example, a reduction of 16 hours to 6-8 hours. In at least one example, the shortest enrichment time may be determined by this process.

The Bayesian enrichment optimization process may be expanded to include particular microbial species or genera. For example, the optimization process may include, but not be limited to, enrichment conditions specific for pathogenic bacterial genera such as Listeria, Staphylococcus, Escherichia, and similar pathogenic bacterial genera.

In block 312, test results and related data are analyzed and qualitative and/or quantitative assessments of the test results and related data are provided. In at least one example, the test results are analyzed by cloud server 216. In at least one example, local or remote computing devices may be used to analyze the data. The qualitative assessment of the test results and related data may include the reliability or accuracy of the detection. The quantitative assessment of the test results and related data may include the time to obtain test results.

In block 316, responsive to the qualitative and/or quantitative assessments of the test results and related data, the test parameters are adjusted to optimize or modify the test parameters and improve performance of the system. For example, module 120 or cloud server 216 may optimize the test parameters in response to the qualitative and/or quantitative assessments. The flow returns to block 304 where subsequent tests are performed using the adjusted or updated test parameters. In at least one example, the foregoing process is repeated for each test, and based on the qualitative and/or quantitative assessments, the test parameters are adjusted to optimize or modify the test parameters and improve the performance of the system.

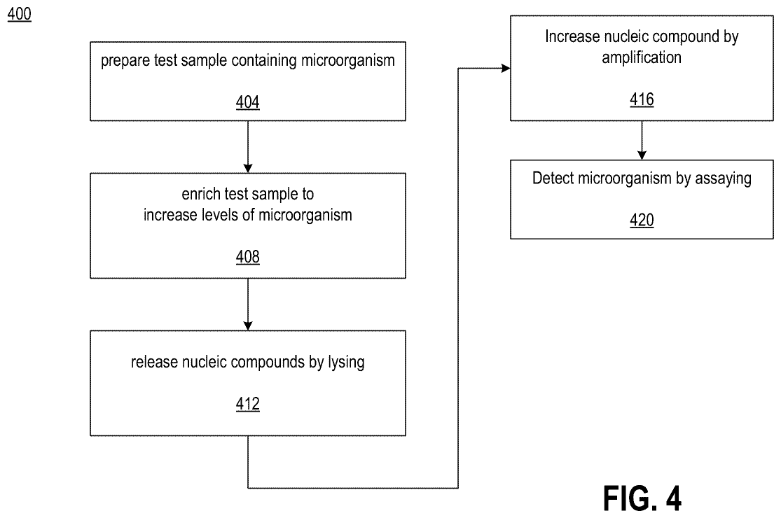

FIG. 4 illustrates a flow diagram of a method 400 of conducting tests in accordance with at least one example. The order of various blocks shown here can be modified. For example, some blocks may be performed simultaneously. In at least one example, the various blocks shown here can be implemented in hardware, software, or a combination of them. In block 404, a test sample (e.g., sample 212) is prepared. The sample may be contaminated with the microorganism. In block 408, the sample is enriched by diluting the sample with a liquid enrichment medium at a first ratio of the sample to the diluent and incubating the diluted sample for a first time period to allow levels of the microorganism to increase.

In block 412, the enriched sample is lysed with a lysis solution at a second ratio of the enriched solution to the lysis solution to break down cells of the microorganism and to release nucleic compounds of the microorganism. The enriched sample may be lysed in microorganism test module 204, in at least one example. In block 416, the nucleic compounds are amplified to increase the number of nucleic compounds. In at least one example, the nucleic compounds may be amplified in microorganism test module 204. In block 420, the sample is assayed to detect the presence or absence of the microorganism. In at least one example, the sample may be assayed in microorganism test module 204.

In at least one example, a test sample is made using the following method. A first mixture containing a sample and suitable amount of phosphate-buffered water is blended. A second mixture is made by adding Bolton antibiotic additive and lysed horse blood to a Bolton broth. A third mixture is made by adding the first mixture and the second mixture, and the pH of the third mixture is adjusted to approximately 7.5. The third mixture is rotated in a centrifuge to separate the test sample from the remaining mixture. The test sample is separated as pellet.

In at least one example, the test sample is enriched by incubating the sample at 37 degrees Celsius for a period of 4 hours or at 42 degrees Celsius for a period of 48 hours, and the test sample is pre-amplified by adding enzymes, reactants, or primers. In at least one example, the presence of the microorganism is detected by the presence of a biomarker or bioluminescence. For example, the enriched sample may be sealed into a culture tube coupled to a portable self-contained analytical laboratory apparatus (e.g., portable bioanalysis station 500A, described below). According to methods described below, an aliquot of the enriched sample may be automatically pipetted into an analytical portion of the self-contained analytical laboratory under software control.

In at least one example, a computer program product comprising a computer readable medium includes computer program logic for the detection of a microorganism in a sample. The computer program logic includes program code to select test parameters for the sample; program code to conduct tests on the sample based on the test parameters and provide test results and related data, wherein the test results indicate the presence or absence of the microorganism; program code to analyze the test results and related data and provide qualitative and quantitative assessments of the test results and related data; program code to adjust the test parameters to optimize or modify the test results; and program code to repeat the tests with the adjusted test parameters and to adjust the test parameters based on analyses of the test results and related data to optimize the test results.

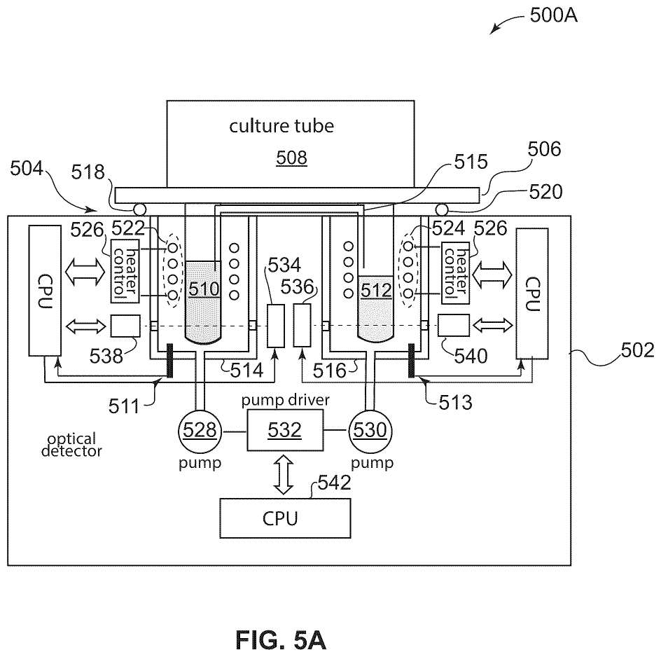

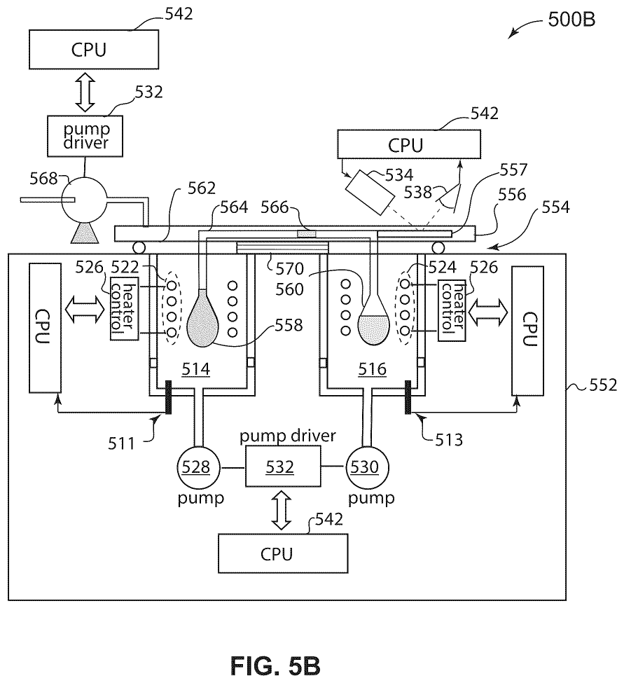

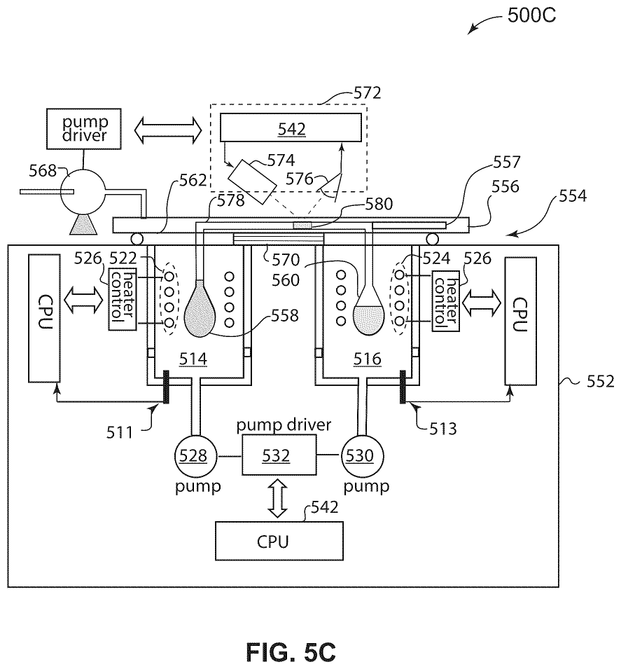

FIG. 5A illustrates a portable bioanalysis station 500A, in accordance with at least one example. In at least one example, portable bioanalysis station 500A is an implementation of system 100 or system 200, described above. In at least one example, portable bioanalysis station 500A comprises base unit 502, providing automated aseptic processing of biological samples suspected of containing pathogenic and/or nonpathogenic microorganisms. In at least one example, base unit 502 may be an implementation of platform 104 or 204, shown in FIGS. 1 and 2, and described above. In at least one example, base unit 502 comprises docking port 504 for attachment of a sample container lid 506. In at least one example, sample container lid 506 is attached to sample container 508. In at least one example, sample container 508 is a disposable culture tube. In at least one example, sample container 508 is capped by sample container lid 506. In at least one example, the sample container is hermetically sealed by sample container lid 506 when attached.

In at least one example, sample container 508 may contain a liquid growth medium for culturing microorganisms contaminating a biological sample, whereby a biological sample may be a solid or liquid substance including samples of solid food or liquid beverages, human or animal tissues, bodily fluids, etc. Such samples may be suspected of being contaminated or infected by a pathogenic organism. A biological sample may be inoculated into a liquid growth medium contained within sample container 508. After an incubation period, an aliquot of enriched liquid growth medium may be injected into a microfluidic laboratory within sample container lid 506, or within a microfluidic cartridge that is fluidically coupled to sample container lid 506. In at least one example, sample container lid 506 may be configured to enable contents of sample container 508 to be sampled by portable bioanalysis station 500A.

In at least one embodiment, docking port 504 is an implementation of interface 108, shown in FIG. 1 and described above. In at least one example, sample container lid 506 comprises an integrated microfluidic laboratory system. In at least one example, sample container lid 506 comprises reagent tubes 510 and 512, integrated on sample container lid 506. Sample container 508 and sample container lid 506 may be presterilized and assembled under sterile conditions to enable aseptic manipulation of fluids between sample container 508 and reagent tubes 510 and 512. Sample container 508 may be presterilized and charged with a biological growth medium, then assembled with sample container lid 506. The assembly may be packaged in sterile packaging until ready to use. The assembly comprising sample container 508 and sample container lid 506 attached to sample container may be hermetically sealed after introduction of a biological sample to sterile growth medium contained within sample container 508. In at least one embodiment, reagent tubes 510 and 512 extend above sample container lid 506 while sample container 508 is upright (or below sample container lid 506 when sample container 508 is inverted). In at least one example, reagent tubes 510 and 512 are implementations of cartridges or containers 208, described above.

In at least one example, the assembly comprising sample container 508 and sample container lid 506 may be docked as self-contained microfluidic laboratory system to docking port 504 by inverting sample container 508 and inserting reagent tubes 510 and 512 into chambers 514 and 516 as sample container lid 506 engages docking port 504. The self-contained and automated nature portable bioanalysis station 500A (including alternate embodiments 500B and 500C, described below) precludes direct handling of samples and reagents, as well as manual execution of laboratory procedures by trained personnel, mitigating contamination and human error in ensuing analytical procedures to identify pathogenic microorganisms, in addition to increasing throughput and time/cost efficiency.

In at least one example, base unit 502 may be configured to receive sample container 508 in an upright orientation, whereby reagent tubes 510 and 512 extend above sample container lid 506. When docked, reagent tubes 510 and 512 are inserted into and seated within chambers 514 and 516, respectively at the top of base unit 502. In at least one embodiment, reagent tubes 510 and 512 have compliant walls to enable compression and expansion of the volumes of reagent tubes 510 and 512 by pressurization and depressurization of chambers 514 and 516. Expansion of reagent tubes 510 and 512 may create a suction by reduced internal air pressure, enabling a pipetting action for aseptically transferring liquids between sample container 508 and reagent tubes 510 and 512, or between reaction tubes 510 or 512, as described below. A hermetic seal may be formed when gaskets 518 and 520 are compressed by portions of sample container lid 506 when secured to docking port 504.

In at least one example, chambers 514 and 516 may comprise internal or external heating elements 522 and 524, respectively, thermally coupled to reagent tubes 510 and 512. In at least one example, heating elements 522 and 524 may be electrically coupled to heater controller 526. In at least one example, heating elements 522 and 524 comprise resistive heating coils, induction heating coils, or hollow tubing coils for circulation of heated fluids. In at least one example, heating elements 522 and 524 may be disposed within chambers 514 and 516 encircling integrated reagent tubes 510 and 512. In at least one example, heating elements 522 and 524 may be disposed outside of chambers 514 and 516 encircling chamber walls. In at least one example, chambers 514 and 516 may be heated by energizing heating elements 522 and 524 via heater controller 526. Heater controller 526 may be commanded by processor system 542. In at least one example, chambers 514 and 516 may be heated or cooled by introduction of preheated or cooled air or liquid (e.g., heated water) into a jacket (not shown) within their walls. In at least one example, chambers 514 and 516 may receive microwave energy for heating of contents contained with reagent tubes 510 and 512.

In at least one example, chambers 514 and 516 may be actively cooled by circulation of refrigerants or chilled water within coils surrounding chambers 514 and 516 or surrounding reagent tubes 510 and 512.

In at least one example, chambers 514 and 516 may be coupled to air pumps 528 and 530, respectively. In at least one example, air pumps 528 and 530 are reversible so that they may be configured to both pressurize and depressurize chambers 514 and 516. In at least one example, pressure sensors 511 and 513 are included within chambers 514 and 516 to enable monitoring of air pressure within. Pressurization and depressurization of chambers 514 and 516 may cause compression and expansion of reagent tubes 510 and 512, respectively, enabling a pipetting action to be actuated for aseptic transfer of fluids from one tube to another. In at least one example, pressurization or depressurization of chambers 514 and 516 may enable squeezing and expanding compliant walls of reagent tubes 510 and 512, or of compliant sacs within reagent tubes 510/512. The expansion or contraction of tube walls may be performed in concert, enable drawing of aliquots of reaction fluids from reagent tube 510 and deliver the aliquots to reagent tube 512.

In at least one example, an aliquot from sample container 508 comprising enriched growth medium containing one or more (pathogenic) microorganism species or genera may be obtained in an aseptic manner by drawing or pipetting the aliquot by depressurization of chamber 514 by pump, whereby compliant walls of reagent tube 510 may expand, increasing internal volume and decreasing internal air pressure. A negative pressure may ensue within the internal volume of reagent tube 510, thereby creating a suction enabling the aliquot to be transferred from sample container 508, through sample container lid 506, to reagent tube 510. In at least one example, reagent tube 510 may contain a cellular lysis reagent for lysing bacterial cells and releasing intracellular contents such as plasmid DNA and cellular proteins.

For transfer of reacted lysis reagent to reagent tube 512, an aliquot of reacted lysis reagent may be obtained from reagent tube 510 by pressurization of chamber 514, whereby reagent tube 510 is compressed via pump 528, whereby air is pumped into chamber. In at least one example, compliant walls of reagent tube 510 may be squeezed or compressed under the increased pressure, reducing internal volume and increasing internal air pressure of reagent tube 510, enabling a transfer of the aliquot of reacted reagent into microchannel 515. In at least one example, microchannel 515 extends within sample container lid 506 between reagent tubes 510 and 512, fluidically interconnecting reagent tubes 510 and 512. In at least one example, microchannel 515 may extend into both reagent tube 510 and reagent tube 512 via vertical microchannels integrated along the walls of reagent tubes 510 and 512 and coupled to ends of microchannel 515).

The aliquot from a first reaction mixture contained in reagent tube 510 may be drawn into a second reaction mixture contained within reagent tube 512 by suction created within, and/or by positive pressure within reagent tube 510 created by compression. In at least one example, the first reaction mixture may comprise a cell wall or cell membrane lysis enzyme or non-biological compound to lyse microbial (e.g., bacterial) cell walls and/or cell membranes, such as a surfactant. The second reaction mixture may contain a reagent that binds to specific cellular proteins and may be caused to fluoresce, or may contain a polymerase chain reaction (PCR) preparation to amplify specific bacterial plasmid DNA segments (genetic material and intracellular proteins from eukaryotic microorganisms such as fungi and protozoa, as well as viral DNA and RNA sequences may be included as target analytes for pathogen identification). In at least one example, the second reaction mixture may contain a dye marker solution comprising a fluorescent marker dye that binds to specific nucleic acid segments (e.g., nucleic acid sequences) amplified by the PCR preparation. The protein fluorescence or amplification of specific nucleic acid sequences may enable identification of a pathogen species or genus, in at least one example.

In at least one example, to transfer an aliquot of reacted cellular lysis reagent, chamber 516 may be depressurized by via pump 530, whereby pump 530 may pump air out of chamber 516. Expansion of compliant walls of reagent tube 512 may be enabled, causing a suction within reagent tube 512. The suction, due to negative internal pressure, may pull the aliquot along microchannel 515. In at least one example, the aliquot may also be pushed by positive pressure within reagent tube 510, drawing the aliquot of reacted cellular lysis reagent into reagent tube 512.

In at least one example, air pumps 528 and 530 are electrically coupled to pump driver 532. Pump driver 532 may supply operating power to air pumps 528 and 530 and be operable to switch power on and off according to commands received from a peripheral processor or a central processing unit (e.g., processor system 542, described below). In at least one example, pump driver 532 may be operable to invert voltage polarity to air pumps 528 and 530 for pressurization or depressurization of chambers 514 and 516, respectively. In at least one example, separate pumps may be employed for pressurization and depressurization, respectively, of chambers 514 and 516.

In at least one example, base unit 502 comprises optical sources 534 and 536. Optical sources 534 and 536 may comprise narrow band solid state lasers, non-coherent broadband optical sources such as halogen or xenon lamps, white-light lasers, etc. In at least one example, optical sources 534 and 536 are aligned such that light may be shined through optical windows within walls of chambers 514 and 516, respectively. Optical sources 534 and 536 may be aligned with optical detectors 538 and 540, respectively. In at least one example, analytical signals may be detected optically by analytes developed in reagent tube 512 by PCR or biomarker formation. In at least one example, optical sources 534 and 536 may be operable to emit ultraviolet light for fluorescence measurements of the intensity of a fluorescence wavelength emitted by a target analyte upon excitation by a visible or ultraviolet light beam emitted by optical source 534 or 536, and/or visible wavelengths for colorimetric determinations. In at least one example, an optical detection signal may be proportional to an attenuation of a light beam emitted by optical source 534 or 536, by optical absorption of one or more wavelengths of the light beam by a target analyte contained within reagent tube 512.

In at least one example, optical detectors 538 and 540 comprise spectrometers to enable wavelength selection from broadband light sources. In at least one example, optical detectors 538 and 540 comprise photodiodes, phototransistors, photovoltaic cells, thermopiles, or other optical detection devices. In at least one example, optical detectors 538 and 540 are coupled to processor system 542, whereby processor system 542 may command optical detectors 538 and 540, as well as optical sources 534 and 536.

In at least one example, detection of the presence or absence of one or more microorganisms may be detected by non-optical signals. In at least one example, an electrochemical sensor may be employed to detect one or more target analytes by electrochemical activity exhibited by a target analyte. at least one example, target analytes may include small molecule metabolites and proteins having electrochemically active centers that are specific to a bacterial species or genus.

FIG. 5B illustrates portable bioanalysis station 500B, in accordance with at least one example. In at least one example, portable bioanalysis station 500B is an alternative embodiment of portable bioanalysis station 500A. In at least one example, portable bioanalysis station 500B comprises base unit 552, comprising docking system 554. In at least one example, docking system 554 is configured to receive microfluidic cartridge 556. In at least one example, microfluidic cartridge 556 may be an implementation of cartridges 208, described above. In at least one example, docking system 554 includes a clamping mechanism (not shown) to secure microfluidic cartridge 556 to base unit 552. Microfluidic cartridge 556 may comprise microchannels, such as microchannel 564, and interconnected microchambers (not shown). One or more microchambers can contain a liquid sample contaminated or inoculated with a microorganism, as well as analytical reagents. In at least one example, a biological sample may be introduced onto microfluidic cartridge 556 by a syringe or a pipette. In at least one example, a biological sample may be introduced onto microfluidic cartridge 556 by a manual or automated micro pipetting system. Once the biological sample is introduced, microfluidic cartridge may be sealed aseptically and docked onto base unit 552.

In at least one example, docking system 554 is configured to enable aseptic fluidic coupling between microfluidic cartridge 556 and reagent tubes 558 and 560 within chambers 514 and 516, respectively. In at least one example, reagent tubes 558 and 560 are compliant sacs that may expand and contract with depressurization and pressurization of chambers 514 and 516. In at least one example, reagent tubes 558 and 560 are reservoirs of reagent for supplying microfluidic cartridge 556. In at least one example, reagent tubes 558 and 560 may be heated by heating elements 522 and 524, respectively, to preheat reagent contained within. In at least one example, microfluidic cartridge 556 provides microchannels for aseptic transfer of fluids between reagent tubes 558 and 560. In at least one example, reagent tubes 558 and 560 may extend to openings on the bottom surface 562 of microfluidic cartridge 556. Compression of reagent tubes 558 and/or 560 by pressurization of chambers 514 and/or 516 can push reagent into microchannel 564 of microfluidic cartridge 556. In at least one example, an aliquot 566 of reagent may be introduced into microchannel 564 by compression of reagent tube 558. Momentary pressurization of chamber 514 and/or depressurization of chamber 516 may enable movement of reagent into microfluidic cartridge 556. Microfluidic cartridge 556 may comprise one or more reaction chambers such as micro-reaction chamber 557, configured to receive reagents via microchannel 564 and other microchannels on microfluidic cartridge 556 from reagent tubes 558 and 560. Reagents introduced onto microfluidic cartridge 556 may mix with sample within reaction chambers. Aliquot size may be determined by the volume of a micro-reaction chamber on microfluidic cartridge 556 which may be filled by the reagent stream as it passes through the micro-reaction chamber. In at least one example, optical source 534 and optical detector may be optically coupled through micro-reaction chamber 557, providing optical detection of reaction products formed within micro-reaction chamber 557.

In at least one example, portable bioanalysis station 500B may comprise a pump 568 operable to move small volumes of liquid, such as sample and reagent. Pump 568 may be fluidically coupled into microfluidic cartridge 556 when microfluidic cartridge 556 engages docking system 554. Pump 568 may comprise a small peristaltic pump, a diaphragm pump or a piston (e.g., syringe) pump. Pump 568 may also be electrically coupled to pump driver 532. Pump 568 may deliver diluent to microfluidic cartridge 556 to push aliquots of sample and reagent through microchannels. In at least one example, docking system 554 may comprise temperature control device 570 to heat and cool microfluidic cartridge 556. In at least one example, temperature control device 570 may comprise a Peltier element that can provide heating and cooling. In at least one example temperature control device 570 is electrically coupled to heater controller 526.

FIG. 5C illustrates portable bioanalysis station 500C, in accordance with at least one example. In at least one example, portable bioanalysis station 500C is an alternative embodiment of portable bioanalysis station 500B. In at least one example, portable bioanalysis station 500C comprises plug flow detector system 572. In at least one example, plug flow detector system 572 comprises light source 574 and detector 576. In at least one example, light source 574 comprises a solid-state laser or a broadband light source, including incoherent light sources. Detector 576 comprises a photodiode or a phototransistor and may comprise a diffraction grating or prism for wavelength detection, in at least one example. Plug flow detector system 572 may detect passage of liquid plugs of sample or reagent flowing in microchannel 578 within microfluidic cartridge 556. In at least one example, an aliquot of reagent may be injected into microchannel 578 from reagent tube 558 by pressurization of chamber 514. The aliquot of reagent forms plug 580 within microchannel 578, pushed along by a diluent such as water, along microchannel 578, in at least one example. A laminar flow regime established within microchannel 578 may suppress mixing of diluent and reagent plug, thereby preserving plug 580 for the duration of flow. The diluent may be injected into microchannel 578 by pump 568, in at least one example. Plug flow detector system 572 may detect plug 580 by detection via detector 576 of a change in refractive index or by absorption of a wavelength emitted by light source 574. In at least one example, detector 576 comprises a fluorescence detector.

Plug flow detector system 572 may be employed to determine volume of aliquot if the volumetric flow rate of fluid within microchannel 578 is known. In at least one example, plug flow detector system 572 is electrically coupled to processor system 542, which may execute software instructions to determine time passage of plug 580. In at least one example, if the flowrate is preset by programming of pump drive 532 via processor system 542 to drive pump 568 to produce a desired flowrate (e.g., or diluent), plug flow detector system 572 may detect a rise and fall of refractive index. Measuring the time between rise and fall of refractive index by processor system 542 can yield aliquot volume by multiplying the time of passage by the volumetric flow rate, a known quantity.

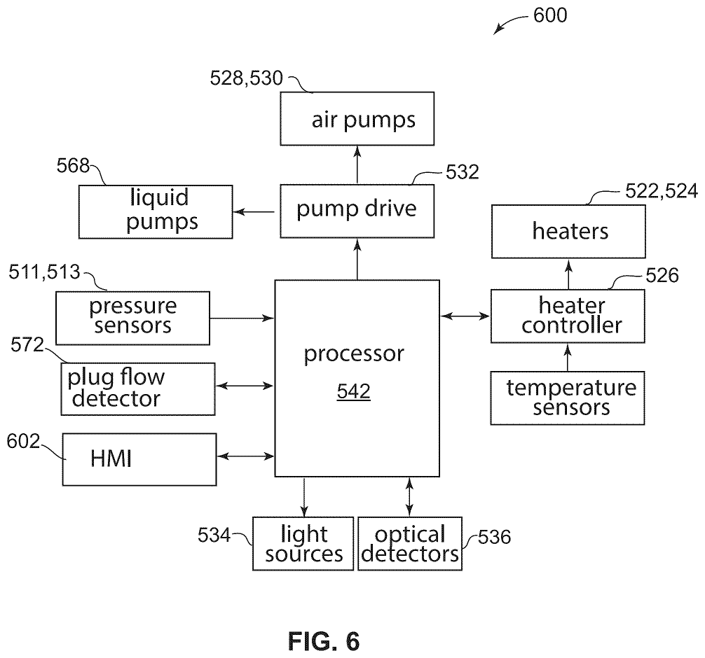

FIG. 6 illustrates a block diagram 600 of an exemplary control configuration for any one of portable bioanalysis stations 500A, 500B or 500C, in at least one example. In at least one example, processor system 542 is in electronic communication with peripherals such as pump drive 532, heater controller 526, optical sources 534 and 536, optical detectors 538 and 540, and pressure sensors 511 and 513. In at least one example, processor system 542 is in communication with plug flow detector system 572 (e.g., light source 574 and detector 576).

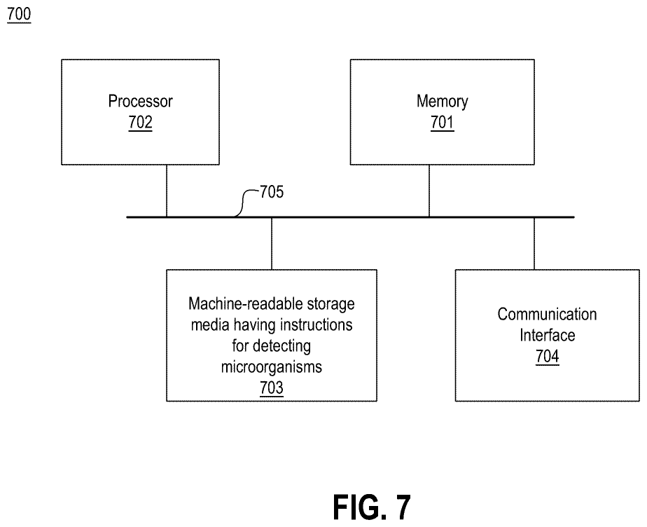

In at least one example, processor system 542 is a stand-alone processor or an embedded processor (e.g., included in a control circuit) within base unit 502 or may be hosted on a physically separate platform from base unit 502. In at least one example, processor system 542 is tasked with peripheral interfacing. In at least one example, processor system 542 may be subordinate to a higher-level processor, such as a CPU associated with module 112 or module 216, shown in FIGS. 1 and 2, respectively, and described above. Modules 112 and/or 216 may be software modules executed by a higher-level processor. In at least one example, processor system 542 may execute software instructions to operate heater controller 526, pump driver 532 optical sources 534 and 536, and optical detectors 538 and 540. Software instructions may be stored in a local memory (e.g., memory 701, FIG. 7), or in a memory associated with module 216 that is hosted on a local server or on an external server or cloud server. In at least one example, current versions of operational software and updates thereof may be retrieved by processor system 542 from an external server hosting module 216, and loaded into local memory.

In at least one example, module 112 may receive test results and/or related data from processor system 542 through interfaces 116, where interfaces 116 may provide one or more physical interfaces between processor system 542 and module 112. Interfaces 116 may comprise a data buffer in local memory or in external memory (e.g., on a server), one or more data registers, or direct connection between I/O pins on processor system 542 and the processor associated with module 112. In at least one example, a processor associated with module 112 may transfer test results and related data to module 120, which may be subordinate to module 112. Module 120 may update one or more test parameters to improve the performance of portable bioanalysis station 500C. The adjusted test parameters may be updated in software stored in local memory associated with processor system 542 or in server memory accessible by processor system 542. In at least one example, module 120 may provide a modified test procedure recipe or calibration data for determining concentrations of a protein measured colorimetrically or by fluorescence, as well as other test results obtained by portable bioanalysis station 500C. The recipe and calibration data may be stored on a server, and transferred to local memory associated with processor system 542 by the higher-level processor associated with module 112.

In at least one example, processor system 542 is coupled to a human-machine interface (HMI) 602 (e.g., interface 224, FIG. 2). In at least one example, interface 224 may permit a user to input data, read data, or modify software instructions by manually changing instructions or parameters (e.g., via dashboard 228).

FIG. 7 illustrates a processor system 700 with machine-readable storage media having instructions that when executed cause a machine to detect the presence or absence of a microorganism and to optimize test parameters. Processes described in various embodiments of the present disclosure may be stored in a machine-readable medium as computer-executable instructions. In at least one example, processor system 700 (also processor system 542 described above) comprises memory 701, processor 702, machine-readable storage media 703 (also referred to as tangible machine-readable medium), communication interface 704 (e.g., wireless or wired interface), and network bus 705 coupled together as shown. In at least one example, the various components of system 700 may be part of processor 702.

In at least one example, processor 702 is a Digital Signal Processor (DSP), an Application Specific Integrated Circuit (ASIC), a general-purpose Central Processing Unit (CPU), or a low power logic implementing a simple finite state machine to perform various processes described herein.

In at least one example, the various logic blocks of processor system 700 are coupled together via network bus 705. Any suitable protocol may be used to implement network bus 705. In at least one example, machine-readable storage medium 703 includes instructions (also referred to as the program software code/instructions) for the detection of microorganism in a sample.

In at least one example, machine-readable storage media 703 is a machine-readable storage media with instructions for the detection of microorganism in a sample (herein machine-readable medium 703) and for providing test results and related data. Machine-readable medium 703 has machine-readable instructions, that when executed, cause processor 702 to perform the method as discussed with reference to various examples.

Program software code/instructions associated with various examples may be implemented as part of an operating system or a specific application, component, program, object, module, routine, or other sequence of instructions, or organization of sequences of instructions referred to as “program software code/instructions,” “operating system program software code/instructions,” “application program software code/instructions,” or simply “software” or firmware embedded in processor. In at least one example, the program software code/instructions associated with processes of various examples are executed by processor system 700.

In at least one example, machine-readable storage media 703 is a computer-executable storage medium 703. In at least one example, the program software code/instructions associated with various embodiments are stored in computer-executable storage medium 703 and executed by processor 702. In at least one example, computer executable storage medium 703 is a tangible machine-readable medium 703 that can be used to store program software code/instructions and data that, when executed by a computing device, causes one or more processors (e.g., processor 702) to perform a process.

The tangible machine-readable medium 703 may include storage of the executable software program code/instructions and data in various tangible locations, including ROM, volatile RAM, non-volatile memory, and/or cache, and/or other tangible memory in at least one example. Portions of this program software code/instructions and/or data may be stored in any one of these storage and memory devices. In at least one example, the program software code/instructions can be obtained from other storage, including, e.g., through centralized servers or peer-to-peer networks and the like, including the Internet. Different portions of the software program code/instructions and data can be obtained at different times and in different communication sessions or in the same communication session.

The software program code/instructions associated with the various embodiments can be obtained in their entirety prior to the execution of a respective software program or application. Alternatively, portions of the software program code/instructions and data can be obtained dynamically, e.g., just in time, when needed for execution. Alternatively, some combination of obtaining the software program code/instructions and data may occur, e.g., for different applications, components, programs, objects, modules, routines or other sequences of instructions or organization of sequences of instructions, in at least one example. Thus, it is not required that the data and instructions be on a tangible machine-readable medium 703 in entirety at a particular instance of time.

Examples of tangible machine-readable medium 703 include but are not limited to recordable and non-recordable type media such as volatile and non-volatile memory devices, read only memory (ROM), random-access-memory (RAM), flash memory devices, floppy and other removable disks, magnetic storage media, optical storage media (e.g., Compact Disk Read-Only Memory (CD ROMS), Digital Versatile Disks (DVDs), etc.), among others. The software program code/instructions may be temporarily stored in digital tangible communication links while implementing electrical, optical, acoustical, or other forms of propagating signals, such as carrier waves, infrared signals, digital signals, etc. through such tangible communication links.

The methods and systems described herein may be deployed in part or in whole through a machine that executes computer software on a server, client, firewall, gateway, hub, router, or other such computer and/or networking hardware. The software program may be associated with a server that may include a file server, print server, domain server, internet server, intranet server, and other variants such as secondary server, host server, distributed server, and the like. The server may include one or more of memories, processors, computer readable media, storage media, ports (physical and virtual), communication devices, and interfaces capable of accessing other servers, clients, machines, and devices through a wired or a wireless medium, and the like. The methods, programs, or codes as described herein and elsewhere may be executed by the server. In addition, other devices required for execution of methods as described in this application may be considered as a part of the infrastructure associated with the server. The server may provide an interface to other devices including, without limitation, clients, other servers, printers, database servers, print servers, file servers, communication servers, distributed servers, social networks, and the like.

Additionally, communication via a wired link or a wireless link may facilitate remote execution of program across the network. The networking of some or all these devices may facilitate parallel processing of a program or method at one or more location without deviating from the scope of the invention. In addition, any of the devices attached to the server through an interface may include at least one storage medium capable of storing methods, programs, code, and/or instructions. A central repository may provide program instructions to be executed on different devices. In this implementation, the remote repository may act as a storage medium for program code, instructions, and programs.

The software program may be associated with a client that may include a file client, print client, domain client, internet client, intranet client, and other variants such as secondary client, host client, distributed client, and the like. The client may include one or more of memories, processors, computer readable media, storage media, ports (physical and virtual), communication devices, and interfaces capable of accessing other clients, servers, machines, and devices through a wired or a wireless medium, and the like. The methods, programs or codes as described herein and elsewhere may be executed by the client. In addition, other devices required for execution of methods as described in this application may be considered as a part of the infrastructure associated with the client.

The networking of some or all these devices may facilitate parallel processing of a program or method at one or more location without deviating from the scope of the invention. In addition, any of the devices attached to the client through an interface may include at least one storage medium capable of storing methods, programs, applications, code and/or instructions. A central repository may provide program instructions to be executed on different devices. In this implementation, the remote repository may act as a storage medium for program code, instructions, and programs.

The methods and systems described herein may be deployed in part or in whole through network infrastructures. The network infrastructure may include elements such as computing devices, servers, cloud servers, routers, hubs, firewalls, clients, personal computers, communication devices, routing devices, and other active and passive devices, modules and/or components as known in the art. The computing and/or non-computing device(s) associated with the network infrastructure may include, apart from other components, a storage medium such as flash memory, buffer, stack, RAM, ROM, and the like. The processes, methods, program codes, and instructions described herein and elsewhere may be executed by one or more of the network elements.

For simplicity and clarity, the full structure and operation of all systems suitable for use with the present disclosure is not being depicted or described herein. Instead, only so much of a system as is unique to the present disclosure or necessary for an understanding of the present disclosure is depicted and described. The remainder of the construction and operation of the disclosed systems may conform to any of the various current implementations and practices known in the art.

Unless specifically indicated or required by the sequence of operations, certain steps in the processes described above may be omitted, performed concurrently or sequentially, or performed in a different order. Further, no component, element, or process should be considered essential to any specific claimed embodiment, and each of the components, elements, or processes can be combined in still other embodiments.

Modifications are possible in the described embodiments, and other embodiments are possible, within the scope of the claims.

Throughout the specification, and in the claims, the term “connected” means a direct connection, such as electrical, mechanical, or magnetic connection between the things that are connected, without any intermediary devices.

The term “coupled” means a direct or indirect connection, such as a direct electrical, mechanical, or magnetic connection between the things that are connected or an indirect connection, through one or more passive or active intermediary devices.

The term “adjacent” here generally refers to a position of a thing being next to (e.g., immediately next to or close to with one or more things between them) or adjoining another thing (e.g., abutting it).

The term “circuit” or “module” may refer to one or more passive and/or active components that are arranged to cooperate with one another to provide a desired function.

The terms “substantially,” “close,” “approximately,” “near,” and “about,” generally refer to being within +/−10% of a target value.

Unless otherwise specified the use of the ordinal adjectives “first,” “second,” and “third,” etc., to describe a common object, merely indicate that different instances of like objects are being referred to and are not intended to imply that the objects so described must be in a given sequence, either temporally, spatially, in ranking or in any other manner.

For the purposes of the present disclosure, phrases “A and/or B” and “A or B” mean (A), (B), or (A and B). For the purposes of the present disclosure, the phrase “A, B, and/or C” means (A), (B), (C), (A and B), (A and C), (B and C), or (A, B and C).

The terms “left,” “right,” “front,” “back,” “top,” “bottom,” “over,” “under,” and the like in the description and in the claims, if any, are used for descriptive purposes and not necessarily for describing permanent relative positions.

Reference in the specification to “an embodiment,” “one embodiment,” “some embodiments,” or “other embodiments” means that a particular feature, structure, or characteristic described in connection with the embodiments is included in at least some embodiments, but not necessarily all embodiments. The various appearances of “an embodiment,” “one embodiment,” or “some embodiments” are not necessarily all referring to the same embodiments. If the specification states a component, feature, structure, or characteristic “may,” “might,” or “could” be included, that particular component, feature, structure, or characteristic is not required to be included. If the specification or claim refers to “a” or “an” element, that does not mean there is only one of the elements. If the specification or claims refer to “an additional” element, that does not preclude there being more than one of the additional elements.

Furthermore, the particular features, structures, functions, or characteristics may be combined in any suitable manner in one or more embodiments. For example, a first embodiment may be combined with a second embodiment anywhere the particular features, structures, functions, or characteristics associated with the two embodiments are not mutually exclusive.

While the disclosure has been described in conjunction with specific embodiments thereof, many alternatives, modifications and variations of such embodiments will be apparent to those of ordinary skill in the art considering the foregoing description. The embodiments of the disclosure are intended to embrace all such alternatives, modifications, and variations as to fall within the broad scope of the appended claims. Where specific details are set forth to describe example embodiments of the disclosure, it should be apparent to one skilled in the art that the disclosure can be practiced without, or with variation of, these specific details. The description is thus to be regarded as illustrative instead of limiting.

We claim:

1. A method for detecting at least one microorganism, the method comprising:
placing a biological sample into a container for detecting a presence or an absence of at least one microorganism via testing of the biological sample within the container as directed by a processor system in accordance with software executing on the processor system;
performing at least one analysis on the biological sample within the container in an automated manner directed by the processor system; and
detecting the presence or the absence of the at least one microorganism as a result of the at least one analysis performed on the biological sample directed by the processor system.

2. The method of claim 1, wherein placing the biological sample into the container comprises inoculating at least a portion of the biological sample into a growth medium contained within the container and hermetically sealing the container with a lid attached to the container, wherein the container is in fluidic communication with a microfluidic laboratory system, wherein the microfluidic laboratory system is integrated into the lid, wherein the lid is attached to the container such that the container is hermetically sealed by the lid, and wherein the microfluidic laboratory system comprises one or more reagent tubes extending above the lid.

3. The method of claim 2, wherein performing the at least one analysis on the biological sample within the container comprises docking the microfluidic laboratory system onto a portable bioanalysis station.

4. The method of claim 3, wherein docking the microfluidic laboratory system onto the portable bioanalysis station comprises inserting a first reagent tube of the one or more reagent tubes into a first chamber within the portable bioanalysis station, and a second reagent tube of the one or more reagent tubes into a second chamber within the portable bioanalysis station, and wherein the first reagent tube of the one or more reagent tubes contains a cellular lysing reagent.

5. The method of claim 4, wherein performing the at least one analysis on the biological sample further comprises transferring a first aliquot comprising the growth medium into the first reagent tube, wherein the first aliquot is drawn into the first reagent tube by a suction created within the first reagent tube, wherein the suction is created by depressurizing the first chamber.

6. The method of claim 5, wherein depressurizing the first chamber comprises pumping air out of the first chamber by a first pump fluidically coupled to the first chamber, wherein the first pump is electrically coupled to a pump driver commanded by the processor system.

7. The method of claim 5, wherein performing the at least one analysis on the biological sample within the container further comprises heating the cellular lysing reagent contained within the first reagent tube by energizing a first heating device thermally coupled to the first reagent tube, wherein the first heating device is commanded by the processor system.

8. The method of claim 6, wherein performing the at least one analysis on the biological sample within the container further comprises transferring a second aliquot comprising the cellular lysing reagent into the second reagent tube of the one or more reagent tubes, wherein the second reagent tube contains a polymerase chain reaction (PCR) preparation or a dye marker solution, wherein the second aliquot is reacted with the PCR preparation or the dye marker solution.

9. The method of claim 8, wherein transferring the second aliquot into the second reagent tube comprises:
pressurizing the first chamber, wherein a first air pressure within the first chamber is increased by the first pump, wherein the first pump is fluidically coupled to the first chamber, wherein the first pump is electrically coupled to the pump driver, and wherein the first pump is commanded by the processor system; or
depressurizing the second chamber, wherein a second air pressure within the second chamber is decreased by a second pump, wherein the second pump is fluidically coupled to the second chamber, wherein the second pump is electrically coupled to the pump driver, and wherein the pump driver is commanded by the processor system.

10. The method of claim 9, wherein detecting the presence or the absence of the at least one microorganism comprises measuring a light signal by an optical detector, wherein the light signal comprises:
an attenuation of a light beam passing through the second reagent tube from a light source, wherein the attenuation is proportional to an absorption of at least one wavelength of the light beam by an analyte in the second reagent tube; or
a fluorescence wavelength emitted by the analyte, wherein the fluorescence wavelength is excited by an optical interaction of the light beam and the analyte, wherein the light source and the optical detector are electrically coupled to the processor system, and wherein the processor system commands the light source.

11. The method of claim 2, wherein placing the biological sample into the container comprises choosing, from among a library of sampling protocols, an enrichment time optimized by a Bayesian optimization procedure.

12. The method of claim 11, wherein the Bayesian optimization procedure selects one or more combinations of parameters that yield a shortest enrichment time, wherein the one or more combinations of parameters comprise any or all of pH, temperature, and supplement concentrations.

13. A method, comprising:
incubating a biological sample contained within a disposable container, wherein the disposable container is fluidically coupled to a microfluidic laboratory system integrated onto a lid configured to attach to the disposable container, and wherein the disposable container is sealed by the lid;
docking the microfluidic laboratory system to a portable bioanalysis station wherein a first reagent tube and a second reagent tube of the microfluidic laboratory system are respectively engaged with a first chamber and a second chamber within the portable bioanalysis station;
transferring a first aliquot comprising the biological sample from the disposable container to the first reagent tube of the microfluidic laboratory system, wherein the first reagent tube contains a cellular lysis reagent;
transferring a second aliquot comprising a mixture comprising the biological sample and the cellular lysis reagent from the first reagent tube to the second reagent tube, wherein the second reagent tube contains a polymerase chain reaction preparation or a biomarker solution; and
detecting a presence or an absence of one or more microorganisms via measuring one or more analyte signal levels developed in the second reagent tube by a detection system in or near the portable bioanalysis station as directed by a processor system substantially in accordance with software executing on the processor system.

14. The method of claim 13, wherein transferring the second aliquot from the first reagent tube to the second reagent tube comprises pressurizing the first chamber of the portable bioanalysis station by pumping air into the first chamber via a first pump commanded by the processor system, and wherein the first reagent tube is compressed such that the cellular lysis reagent flows to the second reagent tube from the first reagent tube.

15. The method of claim 14, wherein transferring the second aliquot from the first reagent tube to the second reagent tube comprises depressurizing the second chamber of the portable bioanalysis station by drawing air out of the second chamber via a second pump commanded by the processor system, and wherein the second reagent tube is expanded such that the cellular lysis reagent flows to the second reagent tube from the first reagent tube.

16. The method of claim 15, wherein transferring the second aliquot from the first reagent tube to the second reagent tube comprises moving the second aliquot through a microchannel on the microfluidic laboratory system, wherein the microchannel interconnects the first reagent tube to the second reagent tube.

17. The method of claim 16, wherein moving the second aliquot through the microchannel on the microfluidic laboratory system comprises injecting a diluent stream into the microchannel.

18. The method of claim 13, wherein transferring the first aliquot comprising the biological sample from the disposable container to the first reagent tube of the microfluidic laboratory system comprises depressurizing the first chamber via a first pump commanded by the processor system, wherein the first reagent tube is expanded to create a negative pressure within the first reagent tube, and wherein the first aliquot is drawn into the first reagent tube from the disposable container.

19. The method of claim 13, wherein detecting the presence or the absence of the one or more microorganisms via measuring the one or more analyte signal levels by the detection system in or near the portable bioanalysis station comprises measuring an intensity of an optical signal or an electrical signal from a non-optical sensor.
